(19) United States
(12) Patent Application Publication
(10) Pub. No.: US 2024/0148509 A1
(43) Pub. Date: May 9, 2024

(54) CONNECTOR FOR OSSEOINTEGRATION PROSTHESIS AND IMPLANT SYSTEM

(71) Applicant: International Prosthetics, Inc., Holbrook, NY (US)

(72) Inventor: Eric J. Schatz, Holbrook, NY (US)

(73) Assignee: International Prosthetics, Inc., Holbrook, NY (US)

(21) Appl. No.: 18/500,560

(22) Filed: Nov. 2, 2023

Related U.S. Application Data
(60) Provisional application No. 63/382,514, filed on Nov. 5, 2022.

(51) Int. Cl. A61F 2/30; A61M 39/10

(52) U.S. Cl. CPC A61F 2/30; A61M 39/10; A61F 2002/30329; A61M 2039/1033

(57) ABSTRACT

A connection device for connecting an implant anchored in bone with an external prosthesis component, such as a limb prosthesis, prosthetic elbow or finger, includes a main housing, having a first attachment connector portion for attachment to the implant system and a second attachment portion for attachment to the prosthesis component. The device further includes a safety mechanism to protect the implant system from high mechanical forces, including rotational forces or bending forces. The safety mechanism includes a rotational force release mechanism with a first component including a ring unit having an inner surface with at least one depression and a second component including at least one raised unit urged into contact with the recession by a fitting.

### CROSS REFERENCE TO RELATED APPLICATION

The present application claims the benefit under 35 U.S.C. § 119 of U.S. Provisional Patent Application No. 63/382,514, filed on Nov. 5, 2022, the contents of which are incorporated herein by reference.

### BACKGROUND OF THE DISCLOSURE

### 1. Field of the Disclosure

The present disclosure relates to a recent surgical procedure that is gaining traction for upper and lower extremity amputees known as Osseointegration. More particularly, it relates to a direct attachment of an external prosthesis to the skeleton through surgical implantation of an intermedullary device. This eliminates the need for a socket to provide stability of the prosthesis and many of the problems that are associated with it.

### 2. Description of the Related Art

Conventional prostheses are shell prostheses that use a socket external to the amputated limb stump to connect to the prosthesis. The inability of the socket to evenly distribute load bearing to such prostheses makes it difficult to meet biomechanical requirements and often leads to skin inflammation and even ulceration due to uneven local forces and friction. In addition, because the receiving cavity is closed, local sweating and malodor are often caused, and the life quality of a patient is seriously influenced. Many disadvantages of shell prostheses have called for clinical and scientific researchers to propose a more biomechanical, friendly, and convenient prosthesis design as soon as possible.

Osseointegration was first started in Sweden in the late 1950's by Dr. Per-Ingvar Brånemark through the use of dental applications. This technique was then applied to facial prostheses such as ears, noses, and hearing aids, as well as subsequent joint replacements in the hand, and silicone prosthetic attachments for thumbs and fingers. However, in the 1990's, Brånemark's team at the Sahlgrenska University Hospital and Integrum AB in Gothenburg performed the first osseointegration procedures for both lower and upper-limb amputees. Since then, thousands of amputees have undergone the procedure in facilities all around the world. Thus, there are two forms of prostheses for any and/or all applications. Therefore, an amputee can utilize a traditional prosthesis, one with a socket, and a prosthesis attached to the body by osseointegration.

There are two primary types of osseointegration procedures currently available for amputees, OPRA and ILP. OPRA was pioneered by Brånemark and his team in Sweden. A competing system known as ILP has also been developed by Orthodynamics GMbH, Lübeck, Germany. Some patients may require more than one surgery. All osseointegration procedures require time for the bone and residual limb to fully heal and strengthen around the metal implant. OPRA is a system that uses a screw shaped prosthesis design, wherein the implant length within the body is relatively short (e.g., 80 mm). Patients are not able to start wearing a prosthesis fully unsupported on their abutment for 6-12 months. ILP is a system that uses a press fit prosthesis design, with an alloy rod and a 3D tripod surface structure having a longer implant length (e.g., 140-180 mm). The healing process is quicker—some patients can start putting full weight on their prosthesis in as soon as 6 weeks (i.e., about 1 and a half months). During rehabilitation the patient will learn to use his/her new prosthesis with the aid of crutches. Rehabilitation can take 3-5 weeks, depending on the patient and whether the surgery was for transfemoral or transtibial amputation. There are no issues with getting the abutment wet, so bathing and swimming are not an issue.

There are two primary companies that distribute an osseointegration implant, Osseointegration Group of Australia (OGA) and Integrum. A prosthetic connector is used to join the prosthesis to the end of the implant. This is a specially made device to protect the implant from excessive loads generated when walking. The loads on an upper extremity implant are far less than a lower extremity implant so its design characteristics should be significantly different. A lower extremity prosthetic connector is too big and heavy to utilize for an upper extremity prosthesis. Furthermore, there is no prosthetic connector made specifically for an upper extremity implant. OGA and Integrum do not make a prosthetic connector that is specific for an upper extremity implant.

Most prosthetics companies now design prostheses for those who have undergone osseointegration. Connection to the abutment is easy, and most amputees report being able to attach or remove their prosthesis in 30 seconds. There is some minor care required for the abutment. The area of skin surrounding the abutment (also known as a stoma) needs to be regularly cleaned.

There is a need for a prosthetic connector that can be used with an upper extremity osseointegration implant.

### SUMMARY OF THE DISCLOSURE

The prosthetic connector disclosed herein is designed to be used with an osseointegration implant, amongst others known and still unknown to the art.

In general, an embodiment of the disclosure is directed to a prosthetic connector that can be used with an upper extremity osseointegration implant. The connector is lightweight and has a short build height so it can fit cosmetically between the implant and the prosthesis. Its conical shape of the main body and locking pin create a toggle-free connection between the implant connector and prosthetic-side connector. The raised ribs ensure a consistent positioning between the limb and prosthesis. The components are 3D printed out of Nylon 11 utilizing an SLS (Selective Laser Sintering) 3D printer. Use of this material aids in reducing vibrations transmitted to the implant (which can be very painful) and provides good impact resistance.

The user can utilize a securely attached male connector fastened to the implant. The male connector can mate with the female connector that is integrated into the prosthesis. The user can utilize a tapered pin with a threaded top to fasten the male connector to the female connector. The female connector can be incorporated into various types of prostheses. This enables the user to change prostheses without changing the male connector attached to the implant.

### BRIEF DESCRIPTION OF THE DRAWINGS

FIGS. 1A, 1B, and 1C are illustrations and a perspective view of the Osseointegration Group of Australia Osseointegration Prosthetic Limb (OGAP-OPL).

FIG. 2 is an illustration and perspective view of Integrum's OPRA Implant System.

FIG. 3 is an illustration of an X-Ray comparing a traditional prosthesis to osseointegration prosthesis.

FIG. 4 is an illustration of a table of Osseointegration implants in use throughout the world.

FIG. 5 is a perspective view of a prosthetic connector according to the present disclosure.

FIGS. 6A1 to the left and 6A2 to the right, 6B1 to the left and 6B2 to the right, and 6C1 to the left and 6C2 to the right are side view, front view, and rear view, respectively, illustrations of the prosthetic connector of FIG. 5 according to the present disclosure.

FIGS. 7-8 are illustrations of a patient with an upper extremity osseointegration implant according to the present disclosure.

FIG. 8 is also an illustration of an upper extremity osseointegration implant utilizing the Implant-Side Male Connector according to the present disclosure.

FIG. 9 is an illustration of an upper extremity osseointegration implant and passive-type arm prosthesis utilizing the prosthetic connector according to one embodiment of the present disclosure.

FIG. 10 is a close-up (bird's eye) view illustration of an upper extremity osseointegration implant utilizing the Implant-Side Male Connector according to the present disclosure.

FIG. 11 is an illustration of an upper extremity osseointegration implant and prosthesis utilizing the prosthetic connector according to one embodiment of the present disclosure.

FIG. 12 is an illustration of an upper extremity osseointegration implant and prosthesis utilizing the prosthetic connector according to another embodiment of the present disclosure.

FIGS. 13A, 13B, and 13C are side view, front view, and rear view, respectively, illustrations of the prosthetic connector according to another embodiment of the present disclosure.

FIGS. 14A, 14B, and 14C are side view, front view, and rear view, respectively, illustrations of an adaptor for the prosthetic connector according to one embodiment of the present disclosure.

A component or a feature that is common to more than one drawing is indicated with the same reference number in each of the drawings.

### DETAILED DESCRIPTION OF THE DISCLOSURE

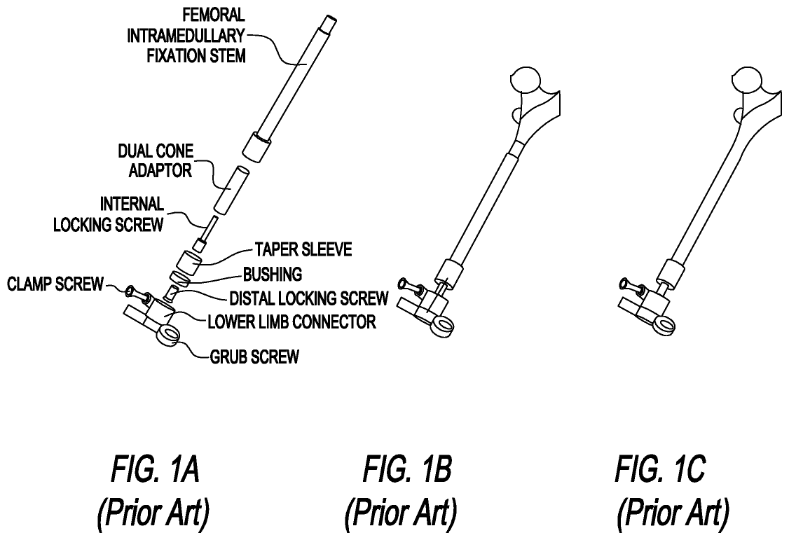

FIGS. 1 through 4 describe the prior art.

In FIGS. 1A, 1B, and 1C, illustrations and a perspective view of the Osseointegration Group of Australia Osseointegration Prosthetic Limb (OGAP-OPL) are shown, according to one embodiment. The first implant utilized for single-stage implantation (i.e., immediate stoma creation) was the Osseointegrated Prosthetic Limb (OPL, Osseointegration International/Permedica SpA, Italy), designed by Dr. Munjed Al Muderis. Also, a press-fit implant, it employs a titanium stem with a macroporous surface coating that facilitates bone ingrowth. The typical length is 140 mm in the femur, similar to the ILP. In patients with adequate bone, weight-bearing exercises often start immediately after surgery. Dr. Al Muderis and his group have implanted over 750 OPLs in the last several years in the femur and tibia. To use this implant in the USA, a humanitarian device exemption needs to be filed with the FDA prior to surgery. A similar system, the Osseointegrated Femur and Tibia (OTN Implant BV, the Netherlands), was developed and is in use in the Netherlands and other parts of Europe. Other implants with very early results include the Percutaneous Osseointegrated Prosthesis (DJO Orthopedics, Austin, TX, USA) under clinical trial and the Compress device (Zimmer Biomet, Warsaw, IN, USA), primarily used for tumor endoprosthetic reconstruction but modified with a transcutaneous prosthetic adaptor.

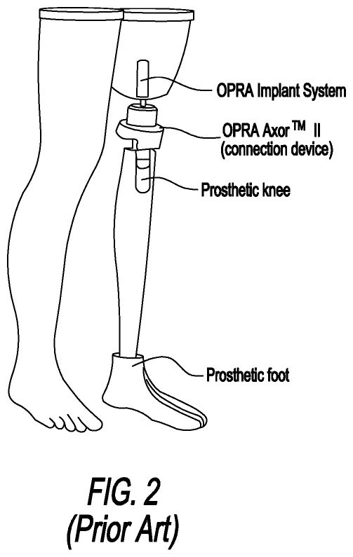

In FIG. 2, an illustration and perspective view of Integrum's OPRA Implant System is shown, according to one embodiment. The OPRA™ Implant System is a bone-anchored prostheses system based on osseointegration, where the prostheses are directly attached to the bone and therefore avoiding the use of a socket. The system consists of three parts; an anchoring element (the Fixture) and a skin penetrating connection (the Abutment), secured with a screw (the Abutment Screw). The unique design ensures the protection of the patient by avoiding bone fractures from accidental loads. The system has been used by hundreds of amputees since the world's first surgery in 1990 and has been proven in several clinical studies. The OPRA Implant System consists of seven titanium or titanium-alloy parts that are implanted during two surgeries. These parts allow a prosthesis to attach directly to the femur (thigh bone). In the first surgery (Stage 1), the fixture is implanted in the femur (thigh bone). Healing time for this surgery is about 6 months. During this time, the bone grows onto the fixture to anchor it in the femur. The hospital stay will last 1 to 3 days, and the patient will be discharged with the appointments he/she needs before the second surgery. Physical therapy is not needed during the 6-month recovery period. Immobilization is preferred in order to optimize healing. After the healing is complete, the abutment is attached to the fixture in the second surgery. The abutment extends outside the skin to attach to the prosthetic. Again, the hospital stay will be 1 to 3 days, and the patient will be discharged with appointments in place, including virtual visits for convenience. The OPRA Axor prosthetic attaches to the abutment outside of the skin installed in the second surgery. It acts as a safety connection between the implant and the prosthesis. It is designed to prevent damage if the implant is overloaded. If an overload occurs, the Axor twists the prosthesis to protect the implant from damage. After six months, the patient will be ready to start rehabilitation with the prosthetic.

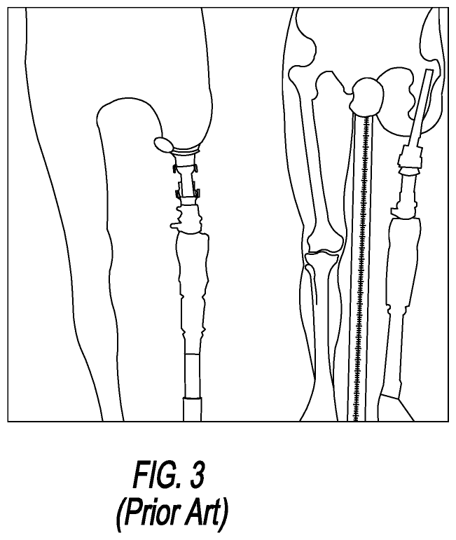

In FIG. 3, a standing radiograph (X-ray) of a patient with a short residual femur in a socket (left) and with an osseointegration implant (right) utilizing an Osseointegrated Prosthetic Limb (OPL) system are shown, according to one embodiment. There is no universal method for loading the prosthesis in the postoperative period. The decision should be patient-specific and based on the quality of bone encountered in the Operating Room (OR), the appearance of the bone and implant on XR/CT, the strength and balance of the patient, and the risk of falling while adapting to a new prosthesis. The screw type implants are gradually loaded immediately after the second surgery because the 3-6-month latency period allows for osseointegration. A short trainer prosthesis is utilized initially, with 20-kg loading performed for 30 min. twice a day and gradually increasing to full body weight at ˜10 kg/wk. Any pain rated greater than a 5 on the visual analog scale warrants an evaluation to ensure fracture has not occurred. Prosthetic gait training then begins around 12 weeks in the presence of a therapist. Walking is initiated with two crutches on flat surfaces for 1-2 hours a day, then progressed to full prosthetic use in 4-6 weeks.

For press-fit implants contacting a long segment of diaphyseal bone with normal density, gradual loading can begin immediately following surgery with the goal to attach a prosthetic leg at six to 10 weeks. Reasons to restrict weight bearing to allow initial osseointegration include thin cortical bone, osteopenic bone, shorter implant contact length, a cortical crack encountered during implantation, and predominantly metaphyseal fit (common in the tibia). At the surgeon's discretion, an initial period of rest without any loading may be advisable. Like the screw fit implants, the press-fit implant is loaded through a short trainer prosthesis, which limits the gravitational pull of the implant and early loosening or dislodgement. An example loading protocol applies 20 lbs. for 10-15 min., 4-6 times per day, with a gradual increase of 5 lbs. per day or every other day. Any pain slows down or halts the progression and imaging is obtained. Once the patient can apply over half body weight through the prosthesis, a prosthetic leg can be attached (FIG. 3). Walking then is protected with crutches or a walker for the next 6 weeks, and gait training is initiated with a physical therapist. For patients with a transfemoral amputation, utilization of a cane or crutch for long-term ambulatory assistance is not considered a failure of treatment, while nearly all transtibial amputees will walk unaided.

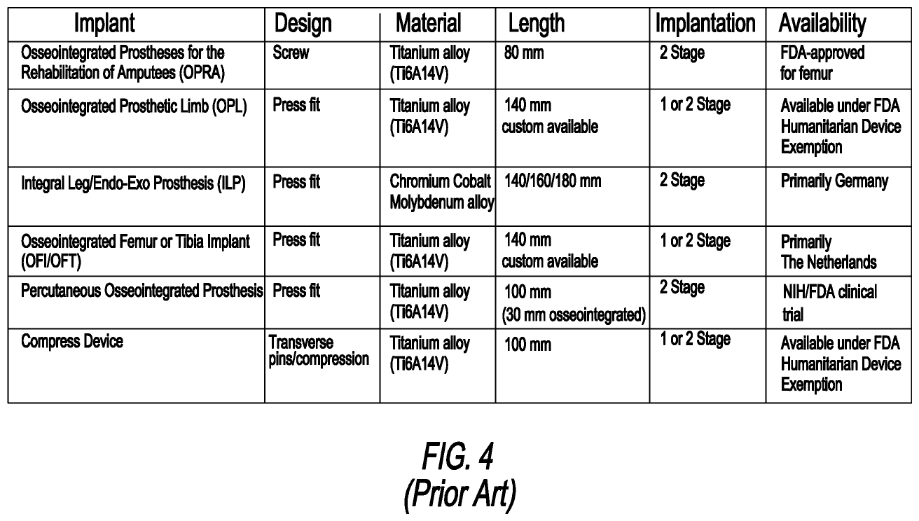

In FIG. 4, an illustration of a table of Osseointegration implants in use throughout the world is shown, according to one embodiment.

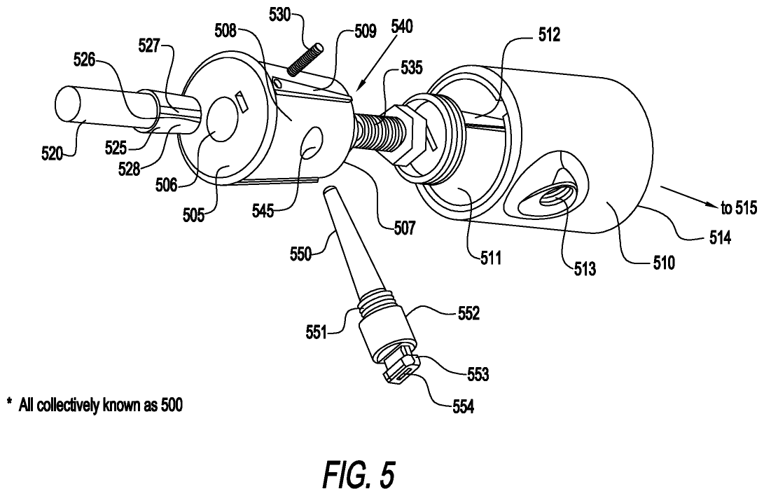

In FIG. 5, a perspective view of a prosthetic connector according to the present invention is shown, according to one embodiment. A Prosthetic Connector 500 consists of two parts, an Implant-Side Male Connector 505 and a Prosthetic-Side Female Connector 510.

An Implant-Side Male Connector 505 may include a proximal interior 506, a threaded distal end 507 (obscured), a proximal exterior 508, raised, tapered ribs 509, a set screw 530, a mounting bolt 535, a hole 540 (obscured), a tapered hole 545, and a tapered pin 550. A tapered pin 550 may include a male thread 551, a head 552, a gripping surface 553, and a rectangular slot 554. A Prosthetic-Side Female Connector 510 may include a proximal interior 511, recessed, tapered channels 512, a female thread 513 and a distal end 514 (obscured) and a tapered hole 545 (obscured). These two connectors mate together to form a rigid connection between a prosthesis 515 (not shown) and an implant 520. An implant 520 may include an implant abutment 525, a threaded distal abutment end 526, a slot 527, and a cone 528.

The user attaches the Implant-Side Male Connector 505 to an implant abutment 525 at a threaded distal abutment end 526 of the implant 520. The threaded distal abutment end 526 of the implant abutment 525 has a conical shape and a slot 527 running longitudinally on the surface of the cone 528. The proximal interior 506 of the Implant-Side Male Connector 505 mates with the conical implant abutment 525. A set screw 530, in the Implant-Side Male Connector 505 is fastened into the slot 527 of the implant abutment 525. This prevents the Implant-Side Male Connector 505 from rotating about the long axis of the implant 520. A mounting bolt 535 is passed through a hole 540 (obscured) in a threaded distal end 507 (obscured) of the Implant-Side Male Connector 505 and fastened into the threaded distal abutment end 526 of the implant abutment 525. This prevents the Implant-Side Male Connector 505 from moving in a longitudinal direction relative to the implant 520. The Implant-Side Male Connector 505 remains on the implant 520 at all times so it can be used with various types of prostheses. The proximal exterior 508 of the Implant-Side Male Connector 505 is conical with raised, tapered ribs 509. The proximal interior 511 of the Prosthetic-Side Female Connector 510 consists of recessed, tapered channels 512 to accept the shape of the Implant-Side Male Connector 505. These tapered mating ribs and/or channels ensure that a prosthesis 515 (not shown) is always donned in a consistent orientation relative to an implant 520. This consistent orientation is crucial for proper function of the various prostheses utilized. The conical shape and tapered ribs and/or channels ensure a tight, toggle-free connection when mated to the Prosthetic-Side Female Connector 510. A tapered through hole 545 is present in both connectors 505 and 510, perpendicular to the long axis of an implant 520. This tapered hole 545 accepts a tapered pin 550 to fasten the Implant-Side Male Connector 505 to the Prosthetic-Side Female Connector 510. A male thread 551 at the head 552 of the tapered pin 550 fastens into a female thread 513 in the Prosthetic-Side Female Connector 510. Above the male thread 551 is a gripping surface 553 to enable the user to tighten the pin 550 into the Prosthetic-Side Female Connector 510. It additionally features a rectangular slot 554 that accepts a broad, flathead screwdriver for added assistance for application or removal of the tapered pin 550 from the Prosthetic-Side Female Connector 510. The distal end 514 of the Prosthetic-Side Female Connector 510 can embody various shapes to enable connection to prosthetic devices such as a body-powered elbow, an externally powered elbow, a passive elbow and/or a quick disconnect fitting, to enable attachment of various terminal devices such as, e.g., a swim paddle.

In another embodiment, it is proposed to integrate a breakaway feature between the Prosthetic-Side Female Connector 510 and a prosthesis 515 (not shown). This will be a safety feature built into the Prosthetic-Side Female Connector 510 to ensure high bending or rotational forces at the prosthesis 515 (not shown) are not transmitted to the implant 520.

In the various views and in the description herein, like components are represented by the same and like reference numerals. For example, 505 and 605 are the same and like components.

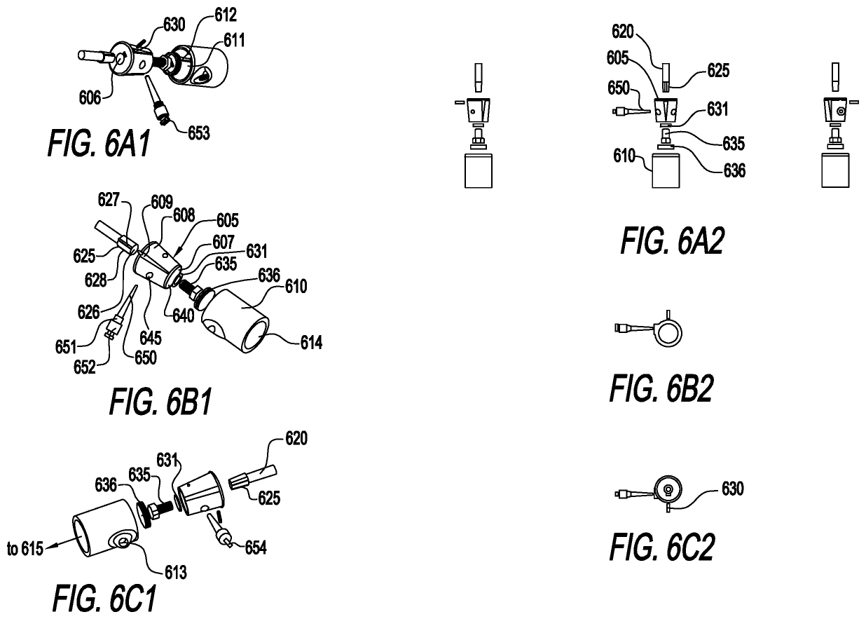

In FIGS. 6A1 to left and 6A2 to the right, 6B1 to the left and 6B2 to the right, and 6C1 to the left and 6C2 to the right, side view, front view, and rear view, respectively, illustrations of the prosthetic connector of FIG. 5 are shown, according to one embodiment. A Prosthetic Connector 600 consists of two parts, an Implant-Side Male Connector 605 and a Prosthetic-Side Female Connector 610.

An Implant-Side Male Connector 605 may include a proximal interior 606, a threaded distal end 607, a proximal exterior 608, raised, tapered ribs 609, a set screw 630, a lock washer 631, a mounting bolt 635, a bolt cap 636, a hole 640, a tapered hole 645, and a tapered pin 650. A tapered pin 650 may include a male thread 651, a head 652, a gripping surface 653, and a rectangular slot 654. A Prosthetic-Side Female Connector 610 may include a proximal interior 611, recessed, tapered channels 612, a female thread 613, a distal end 614, and a tapered hole 645. These two connectors mate together to form a rigid connection between a prosthesis 615 (not shown) and an implant 620. An implant 620 may include an implant abutment 625, a threaded distal abutment end 626, a slot 627, and a cone 628.

The user attaches the Implant-Side Male Connector 605 to an implant abutment 625 at a threaded distal abutment end 626 of the implant 620. The threaded distal abutment end 626 of the implant abutment 625 has a conical shape and a slot 627 running longitudinally on the surface of the cone 628. The proximal interior 606 of the Implant-Side Male Connector 605 mates with the conical implant abutment 625. A set screw 630, in the Implant-Side Male Connector 605 is fastened into the slot 627 of the implant abutment 625. This prevents the Implant-Side Male Connector 605 from rotating about the long axis of the implant 620. A mounting bolt 635 is passed through a hole 640 in a threaded distal end 607 of the Implant-Side Male Connector 605 and fastened into the threaded distal abutment end 626 of the implant abutment 625. This prevents the Implant-Side Male Connector 605 from moving in a longitudinal direction relative to the implant 620. The Implant-Side Male Connector 605 remains on the implant 620 at all times so it can be used with various types of prostheses. The proximal exterior 608 of the Implant-Side Male Connector 605 is conical with raised, tapered ribs 609. The proximal interior 611 of the Prosthetic-Side Female Connector 610 consists of recessed, tapered channels 612 to accept the shape of the Implant-Side Male Connector 605. These tapered mating ribs and/or channels ensure that a prosthesis 615 (not shown) is always donned in a consistent orientation relative to an implant 620. This consistent orientation is crucial for proper function of the various prostheses utilized. The conical shape and tapered ribs and/or channels ensure a tight, toggle-free connection when mated to the Prosthetic-Side Female Connector 610. A tapered through hole 645 is present in both connectors 605 and 610, perpendicular to the long axis of an implant 620. This tapered hole 645 accepts a tapered pin 650 to fasten the Implant-Side Male Connector 605 to the Prosthetic-Side Female Connector 610. A male thread 651 at the head 652 of the tapered pin 650 fastens into a female thread 613 in the Prosthetic-Side Female Connector 610. Above the male thread 651 is a gripping surface 653 to enable the user to tighten the pin 650 into the Prosthetic-Side Female Connector 610. It additionally features a rectangular slot 654 that accepts a broad, flathead screwdriver for added assistance for application or removal of the tapered pin 650 from the Prosthetic-Side Female Connector 610. The distal end 614 of the Prosthetic-Side Female Connector 610 can embody various shapes to enable connection to prosthetic devices such as a body-powered elbow, an externally powered elbow, a passive elbow and/or a quick disconnect fitting, to enable attachment of various terminal devices such as, e.g., a swim paddle.

In another embodiment, it is proposed to integrate a breakaway feature between the Prosthetic-Side Female Connector 610 and a prosthesis 615 (not shown). This will be a safety feature built into the Prosthetic-Side Female Connector 610 to ensure high bending or rotational forces at the prosthesis 615 (not shown) are not transmitted to the implant 620.

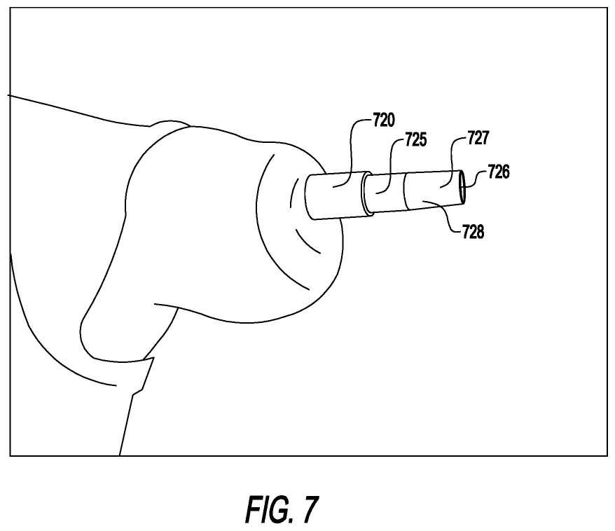

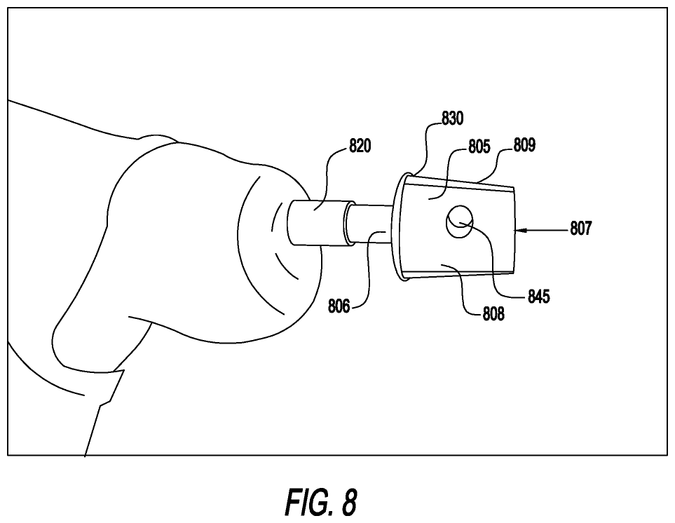

In FIGS. 7-8, illustrations of a patient with an upper extremity osseointegration implant are shown, according to one embodiment. An implant 720 may include an implant abutment 725, a threaded distal abutment end 726, a slot 727, and a cone 728. Two predominant implant designs are currently in use—screw-type and press-fit. They have significant differences regarding surgical technique, rehabilitation, and time to ambulation but ultimately rely on the same principle—the creation of a bone and metal interface that is tightly opposed at the microscopic level. The bone metal interface is similar to that of an uncemented femoral stem in a hip replacement and likewise stable enough to support weight-bearing activity.

The Osseointegrated Prostheses for the Rehabilitation of Amputees (OPRA, Integrum AB, Sweden) is the oldest osseointegration implant and was used in the first osseointegration procedure pioneered by Brånemark in Sweden in 1990, as shown in FIG. 4. Its outer threads screw into the bone like the original technique used in the jaw. The length of the intramedullary portion of the OPRA implant is 80 mm. Using the standard OPRA technique, two surgical procedures are performed 6 months apart to allow adequate implant integration with the host bone. The first procedure implants the threaded intramedullary bone anchor, and the distal soft tissue is fully closed. The second procedure creates a stoma at the skin-implant interface and attaches the transcutaneous abutment to the implanted fixture. A short training prosthesis is then attached, and the patient increases load on the bone until a full leg prosthesis is attached at around 6 weeks. At 4 months, the patients are encouraged to increase prosthetic wear time and at 6 months graduate to independent walking without crutches if possible. Over 500 OPRAs have been implanted according to the company website. It is approved for use in the femur by the US FDA.

The Integral Leg Prosthesis (ILP, previously Endo-Exo Prosthesis, Eska Orthopaedics GmbH, Germany) implant was developed in Germany by Hans Grundei. The design is 140-180 mm in length, significantly longer than the OPRA. The intramedullary canal is prepared via sequential reaming and broaching until a press-fit of the implant with the bone is obtained and a temporary plug is inserted into the distal end of the implant. Approximately 6 weeks later, a circular coring blade is used to open the skin over the abutment to create a stoma. The implant plug is removed, and a dual cone adaptor is inserted percutaneously. Rehabilitation proceeds as tolerated and the permanent prosthetic limb is attached within the first few weeks after the second stage. The change in latency from implantation to stoma creation from 6 months to 6 weeks means the prosthetic leg is attached more quickly than with the OPRA. The implant is widely used in Europe but not in the USA.

The first implant utilized for single-stage implantation (i.e., immediate stoma creation) was the Osseointegrated Prosthetic Limb (OPL, Osseointegration International/Permedica SpA, Italy), designed by Al Muderis, as shown in FIGS. 1A, 1B, and 1C. Also, a press-fit implant, it employs a titanium stem with a macroporous surface coating that facilitates bone ingrowth. The typical length is 140 mm in the femur, similar to the ILP. In patients with adequate bone, weight-bearing exercises often start immediately after surgery. Al Muderis and his group have implanted over 750 OPLs in the last several years in the femur and tibia. To use this implant in the USA, a humanitarian device exemption needs to be filed with the FDA prior to surgery. A similar system, the Osseointegrated Femur and Tibia (OTN Implant BV, the Netherlands), was developed and is in use in the Netherlands and other parts of Europe. Other implants with very early results include the Percutaneous Osseointegrated Prosthesis (DJO Orthopedics, Austin, TX, USA) under clinical trial and the Compress device (Zimmer Biomet, Warsaw, IN, USA), primarily used for tumor endoprosthetic reconstruction but modified with a transcutaneous prosthetic adaptor, as shown in FIG. 4.

Once the implant is inserted in the bone, the muscle, subcutaneous tissue, and skin must be closed around the transcutaneous portion. There is less published literature on the details of this topic, so methods vary. Some surgeons support opposing the bone end as close to the skin as possible without intervening muscle so that the skin adheres to the bone like an antler. Others perform a purse string myoplasty over the bone end and the subcutaneous fat is thinned but the skin maintains a robust vascular supply and can grow around the implant. The skin is more mobile but over time forms a loose seal around the prosthesis. Regardless, once the implant is osseointegrated, the bone/implant interface is seemingly resistant to deep infection, discussed further below, which has allowed the technology to persist.

All implant designs today have smooth polished metal traversing the skin because textured designs lead to a high rate of pain, drainage, and ultimately stoma revisions and have been abandoned. In general, excess soft tissue can impinge on the attached prosthesis or lead to pistoning, drainage, bleeding, inflammation, and increased shear on the skin (which may contribute to superficial infection) so care is taken to remove as much excess tissue as possible around the stoma, as shown in FIGS. 7-8. A plastic surgeon can be helpful with this portion of the procedure, shaping the soft tissue in a smooth contour around the stoma, and ultimately decreasing soft tissue complications.

In FIG. 8, an illustration of an upper extremity osseointegration implant utilizing the Implant-Side Male Connector is shown, according to the present invention. A Prosthetic Connector consists of two parts, an Implant-Side Male Connector 805 and a Prosthetic-Side Female Connector (not shown).

An Implant-Side Male Connector 805 may include a proximal interior 806, a threaded distal end 807, a proximal exterior 808, raised, tapered ribs 809, a set screw 830, a mounting bolt, a hole, a tapered hole 845, and a tapered pin. A tapered pin may include a male thread, a head, a gripping surface, and a rectangular slot. A Prosthetic-Side Female Connector may include a proximal interior, recessed, tapered channels, a female thread, a distal end, and a tapered hole. These two connectors mate together to form a rigid connection between a prosthesis and an implant 820. An implant 820 may include an implant abutment, a threaded distal abutment end, a slot, and a cone.

In another embodiment, it is proposed to integrate a breakaway feature between the Prosthetic-Side Female Connector and a prosthesis. This will be a safety feature built into the Prosthetic-Side Female Connector to ensure high bending or rotational forces at the prosthesis are not transmitted to the implant 820.

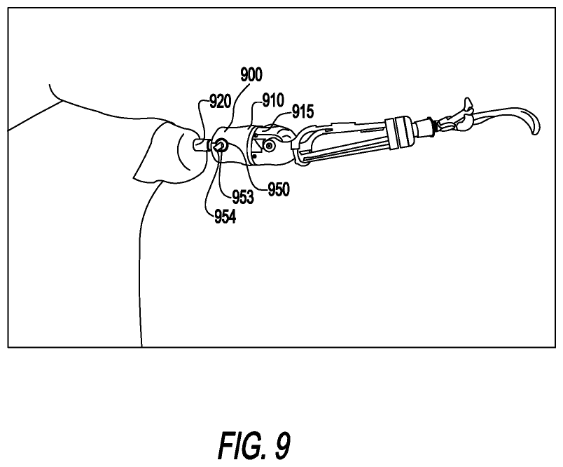

In FIG. 9, an illustration of an upper extremity osseointegration implant and prosthesis utilizing the prosthetic connector is shown, according to one embodiment. A Prosthetic Connector 900 consists of two parts, an Implant-Side Male Connector (not shown) and a Prosthetic-Side Female Connector 910.

An Implant-Side Male Connector may include a proximal interior, a threaded distal end, a proximal exterior, raised, tapered ribs, a set screw, a mounting bolt, a hole, a tapered hole, and a tapered pin 950. A tapered pin 950 may include a male thread, a head, a gripping surface 953, and a rectangular slot 954. A Prosthetic-Side Female Connector 910 may include a proximal interior, recessed, tapered channels, a female thread, a distal end, and a tapered hole. These two connectors mate together to form a rigid connection between a prosthesis 915 and an implant 920. An implant 920 may include an implant abutment, a threaded distal abutment end, a slot, and a cone.

The user attaches the Implant-Side Male Connector to an implant abutment at a threaded distal abutment end of the implant. The threaded distal abutment end of the implant abutment has a conical shape and a slot running longitudinally on the surface of the cone. The proximal interior of the Implant-Side Male Connector mates with the conical implant abutment. A set screw, in the Implant-Side Male Connector is fastened into the slot of the implant abutment. This prevents the Implant-Side Male Connector from rotating about the long axis of the implant. A mounting bolt is passed through a hole in a threaded distal end of the Implant-Side Male Connector and fastened into the threaded distal abutment end of the implant abutment. This prevents the Implant-Side Male Connector from moving in a longitudinal direction relative to the implant 920. The Implant-Side Male Connector remains on the implant 920 at all times so it can be used with various types of prostheses. The proximal exterior of the Implant-Side Male Connector is conical with raised, tapered ribs. The proximal interior of the Prosthetic-Side Female Connector 910 consists of recessed, tapered channels to accept the shape of the Implant-Side Male Connector. These tapered mating ribs and/or channels ensure that a prosthesis 915 is always donned in a consistent orientation relative to an implant 920. This consistent orientation is crucial for proper function of the various prostheses utilized. The conical shape and tapered ribs and/or channels ensure a tight, toggle-free connection when mated to the Prosthetic-Side Female Connector 910. A tapered through hole is present in both connectors and, perpendicular to the long axis of an implant 920. This tapered hole accepts a tapered pin 950 to fasten the Implant-Side Male Connector to the Prosthetic-Side Female Connector 910. A male thread at the head of the tapered pin 950 fastens into a female thread in the Prosthetic-Side Female Connector 910. Above the male thread is a gripping surface 953 to enable the user to tighten the pin 950 into the Prosthetic-Side Female Connector 910. It additionally features a rectangular slot 954 that accepts a broad, flathead screwdriver for added assistance for application or removal of the tapered pin 950 from the Prosthetic-Side Female Connector 910. The distal end of the Prosthetic-Side Female Connector 910 can embody various shapes to enable connection to prosthetic devices such as a body-powered elbow, an externally powered elbow, a passive elbow and/or a quick disconnect fitting, to enable attachment of various terminal devices such as, e.g., a swim paddle.

In another embodiment, it is proposed to integrate a breakaway feature between the Prosthetic-Side Female Connector 910 and a prosthesis 915. This will be a safety feature built into the Prosthetic-Side Female Connector 910 to ensure high bending or rotational forces at the prosthesis 915 are not transmitted to the implant 920.

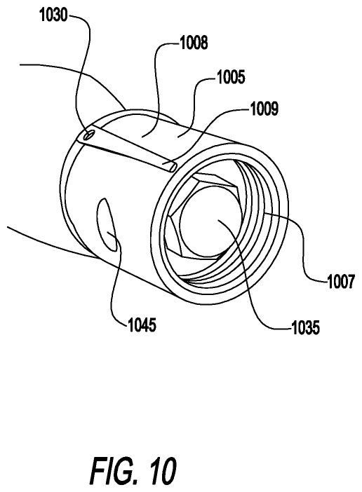

In FIG. 10, a close-up (bird's eye) view illustration of an upper extremity osseointegration implant utilizing the Implant-Side Male Connector is shown, according to the present invention. A Prosthetic Connector consists of two parts, an Implant-Side Male Connector 1005 and a Prosthetic-Side Female Connector (not shown).

An Implant-Side Male Connector 1005 may include a proximal interior, a threaded distal end 1007, a proximal exterior 1008, raised, tapered ribs 1009, a set screw 1030, a mounting bolt 1035, a hole, a tapered hole 1045, and a tapered pin. A tapered pin may include a male thread, a head, a gripping surface, and a rectangular slot. A Prosthetic-Side Female Connector may include a proximal interior, recessed, tapered channels, a female thread, a distal end, and a tapered hole. These two connectors mate together to form a rigid connection between a prosthesis and an implant. An implant may include an implant abutment, a threaded distal abutment end, a slot, and a cone.

In another embodiment, it is proposed to integrate a breakaway feature between the Prosthetic-Side Female Connector and a prosthesis. This will be a safety feature built into the Prosthetic-Side Female Connector to ensure high bending or rotational forces at the prosthesis are not transmitted to the implant.

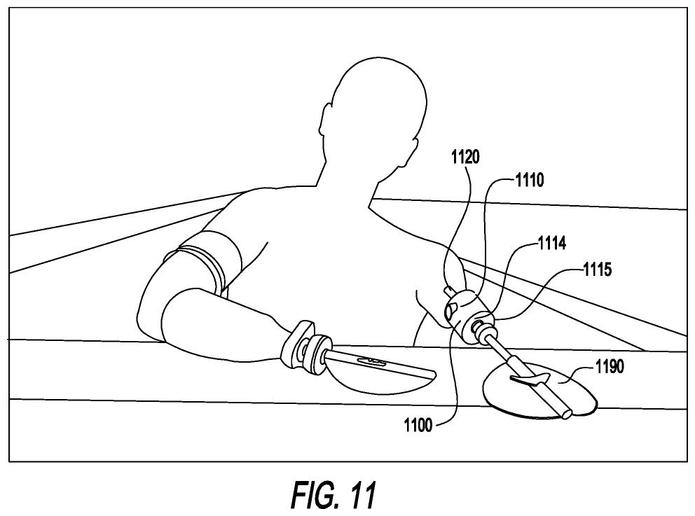

In FIG. 11, an illustration of an upper extremity osseointegration implant and prosthesis utilizing the prosthetic connector is shown, according to one embodiment. The Prosthetic Connector 1100 may include a distal end 1114 of the Prosthetic-Side Female Connector 1110, which can embody various shapes to enable connection to prosthetic devices such as a body-powered elbow, an externally powered elbow, a passive elbow and/or a quick disconnect fitting, to enable attachment of various terminal devices such as, e.g., a swim paddle 1190.

In another embodiment, it is proposed to integrate a breakaway feature between the Prosthetic-Side Female Connector 1110 and a prosthesis 1115. This will be a safety feature built into the Prosthetic-Side Female Connector 1110 to ensure high bending or rotational forces at the prosthesis 1115 are not transmitted to the implant 1120.

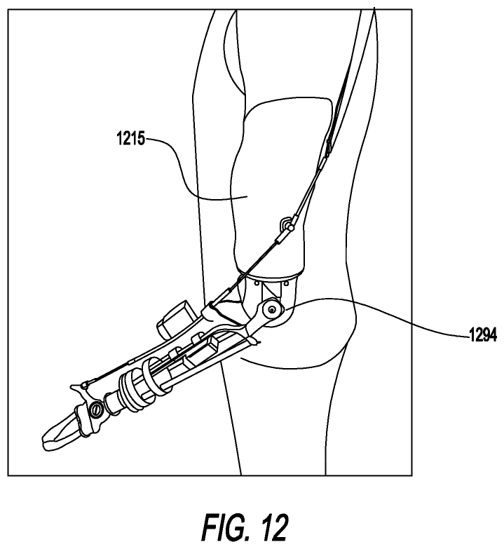

In FIG. 12, an illustration of an upper extremity osseointegration implant and prosthesis utilizing the prosthetic connector is shown, according to another embodiment. The distal end of the Prosthetic-Side Female Connector can embody various shapes to enable connection to prosthetic devices such as a body-powered elbow 1294, an externally powered elbow, a passive elbow and/or a quick disconnect fitting, to enable attachment of various terminal devices such as, e.g., a swim paddle.

In another embodiment, it is proposed to integrate a breakaway feature between the Prosthetic-Side Female Connector and a prosthesis 1215. This will be a safety feature built into the Prosthetic-Side Female Connector to ensure high bending or rotational forces at the prosthesis 1215 are not transmitted to the implant.

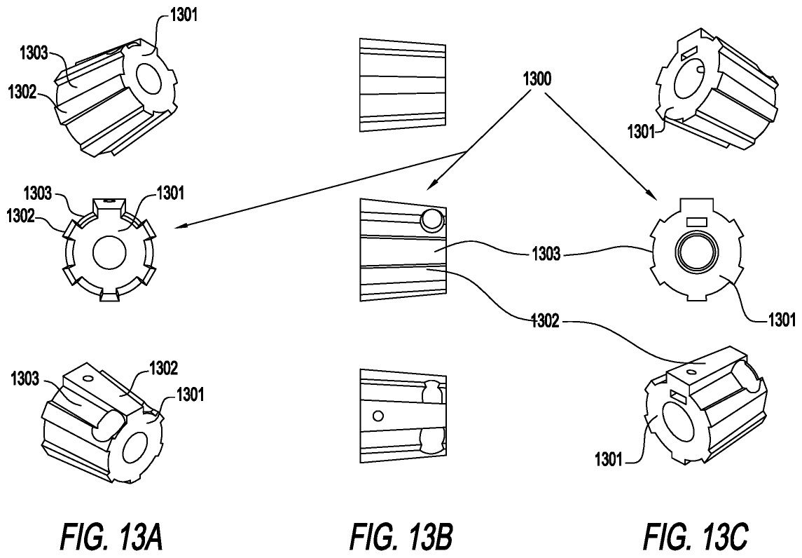

In FIGS. 13A, 13B, and 13C, side view, front view, and rear view, respectively, illustrations of the prosthetic connector are shown, according to another embodiment. The Prosthetic Connector 1300, including the Implant-Side Male Connector 1301 and Prosthetic-Side Female Connector (not shown) may comprise a plurality of raised ribs 1302 and/or recessed channels 1303, accordingly. These raised ribs 1302 and/or recessed channels 1303 may vary in width and depth to ensure only one orientation between the Implant-Side Male 1301 and Prosthetic-Side Female Connectors, of the Prosthetic Connector 1300.

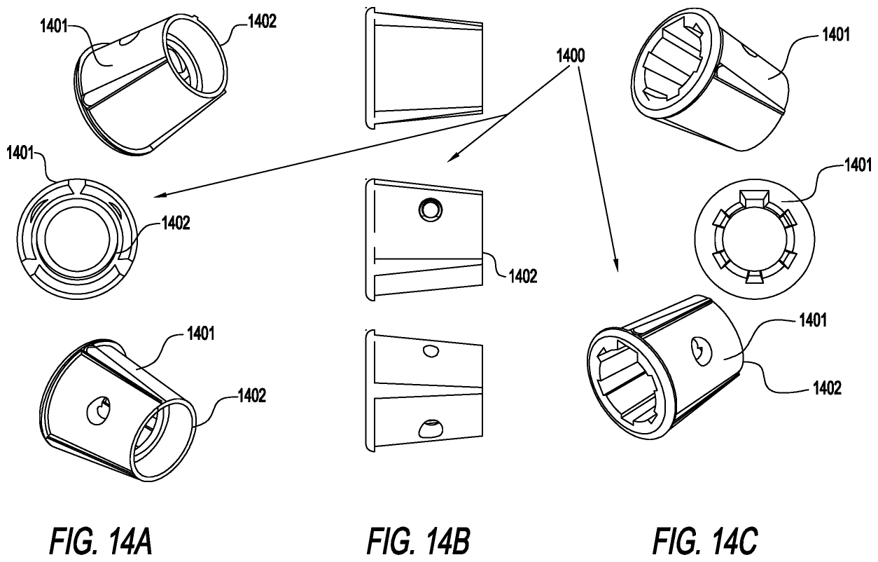

In FIGS. 14A, 14B, and 14C, side view, front view, and rear view, respectively, illustrations of an adaptor for the prosthetic connector are shown, according to one embodiment. If the overall size of the Implant-Side Male Connector 1301 (not shown), as shown and described in FIGS. 13A-C, is reduced, the Prosthetic Connector 1400 may comprise an adaptor piece 1401 and a threaded distal end 1402, utilized so that the smaller Implant-Side Male Connector 1301 may be used with the larger Prosthetic-Side Female Connector (not shown), of the Prosthetic Connector 1400. This will enable use of an improved Implant-Side Male Connector 1301 to be retrofitted to a previous iteration and/or embodiment of the Prosthetic-Side Female Connector.

In another embodiment, a safety mechanism may include a bending force release mechanism with a second attachment portion being pivotable. The safety mechanism may be arranged to limit rotating forces as well as bending forces. A breakaway feature may be between the Prosthetic-Side Female Connector (second connector) and the prosthesis to ensure high bending and/or rotational forces at the prosthesis are not transmitted to the implant. A method for using the system is also disclosed herein.

The techniques described herein are exemplary, and should not be construed as implying any particular limitation on the present disclosure. It should be understood that various alternatives, combinations and modifications could be devised by those skilled in the art. For example, steps associated with the processes described herein can be performed in any order, unless otherwise specified or dictated by the steps themselves. The present disclosure is intended to embrace all such alternatives, modifications and variances that fall within the scope of the appended claims.

The terms “comprises” or “comprising” are to be interpreted as specifying the presence of the stated features, integers, steps or components, but not precluding the presence of one or more other features, integers, steps or components or groups thereof

What is claimed is:

1. A connection device for attaching an implant to a prosthesis, comprising:
a male connector sized, shaped, and configured to attach to an upper extremity implant; and
a female connector sized, shaped, and configured to attach to a prosthesis component,
wherein when said male connector is attached to said upper extremity implant and said female connector is attached to said prosthesis component, a rigid connection is made between said prosthesis component and said upper extremity implant.

2. The connection device of claim 1, wherein said male connector attached to said upper extremity implant includes a proximal interior, a threaded distal end, a proximal exterior, raised, tapered ribs, a set screw, a mounting bolt, a hole, a tapered hole, and a tapered pin.

3. The connection device of claim 2, wherein said tapered pin includes a male thread, a head, a gripping surface, and a rectangular slot.

4. The connection device of claim 2, wherein said threaded distal end of said male connector embodies various portions shaped to enable connection to said female connector.

5. The connection device of claim 1, wherein said female connector attached to said prosthesis component includes a proximal interior, recessed, tapered channels, a female thread, a distal end, and a tapered hole.

6. The connection device of claim 1, wherein said upper extremity implant includes an implant abutment, a threaded distal abutment end, a slot, and a cone.

7. The connection device of claim 1, wherein said distal end of said female connector embodies various portions shaped to enable connection to said prosthesis component.

8. The connection device of claim 2, wherein said male connector further comprises at least a plurality of raised, tapered ribs and at least a plurality of recessed, tapered channels.

9. The connection device of claim 8, further comprising an adaptor sized, shaped, and configured to attach to said male connector.

10. The connection device of claim 9, wherein a threaded distal end of said adaptor embodies various portions shaped to enable connection to said female connector.

11. The connection device of claim 7, wherein said female connector is sized, shaped, and configured to connect to said prosthesis component, and said prosthesis component is selected from the group consisting of a body-powered elbow, an externally powered elbow, a passive elbow and a quick disconnect fitting, and are enabled for attachment of various terminal devices including a swim paddle.

12. A connection device for attaching an implant to a prosthesis, comprising:
a male connector attached to an upper extremity implant and a female connector attached to a prosthesis component,
wherein said male connector attached to said upper extremity implant includes a proximal interior, a threaded distal end, a proximal exterior, raised, tapered ribs, a set screw, a mounting bolt, a hole, a tapered hole, and a tapered pin,
wherein said tapered pin includes a male thread, a head, a gripping surface, and a rectangular slot; and
wherein said female connector attached to said prosthesis component includes a proximal interior, recessed, tapered channels, a female thread, a distal end, and a tapered hole, and
wherein said male connector attached to said upper extremity implant and said female connector attached to said prosthesis component, mate together to form a rigid connection between said prosthesis component and said upper extremity implant, and
wherein said upper extremity implant includes an implant abutment, a threaded distal abutment
end, a slot, and a cone.

13. The connection device of claim 12, wherein said threaded distal end of said male connector embodies various portions shaped to enable connection to said female connector.

14. The connection device of claim 12, wherein said distal end of said female connector embodies various portions shaped to enable connection to said prosthesis component.

15. The connection device of claim 12, wherein said male connector further comprises at least a plurality of raised, tapered ribs and at least a plurality of recessed, tapered channels.

16. The connection device of claim 15, further comprising an adaptor sized, shaped, and configured to attach to said male connector.

17. The connection device of claim 16, wherein a threaded distal end of said adaptor embodies various portions shaped to enable connection to said female connector.

18. The connection device of claim 14, wherein said female connector is sized, shaped, and configured to connect to said prosthesis component, and said prosthesis component is selected from the group consisting of a body-powered elbow, an externally powered elbow, a passive elbow and a quick disconnect fitting, and are enabled for attachment of various terminal devices including a swim paddle.
